(19) United States
(12) Patent Application Publication
(10) Pub. No.: US 2018/0333069 A1
(43) Pub. Date: Nov. 22, 2018

(54) MAGNETIC RESONANCE APPARATUS AND OPERATING METHOD THEREFOR

(71) Applicant: Siemens Healthcare GmbH, Erlangen (DE)

(72) Inventors: Dominik Paul, Bubenreuth (DE); Peter Schmitt, Weisendorf (DE)

(73) Assignee: Siemens Healthcare GmbH, Erlangen (DE)

(21) Appl. No.: 15/984,934

(22) Filed: May 21, 2018

(30) Foreign Application Priority Data
May 22, 2017 (EP) 17172144

(51) Int. Cl. A61B 5/055; G06T 7/00; G01R 33/483

(52) U.S. Cl. CPC A61B 5/055; G06T 7/149; G01R 33/483; G06T 7/0012

(57) ABSTRACT

In a magnetic resonance apparatus and an operating method therefor, an MR image data record is provided to a computer, wherein at least one image of the magnetic resonance image data record is distorted. The computer generates a selection symbol for selecting a measurement volume, wherein the selection symbol is distorted so as to have a rectangular cross section after a distortion correction, and superimposing the selection symbol onto an image of the magnetic resonance image data record.

### BACKGROUND OF THE INVENTION

### Field of the Invention

The present invention concerns magnetic resonance (MR) imaging, and spectroscopy in particular to an MR apparatus having an MR data acquisition scanner that is operable to acquire raw MR data from a subject, and to a method for operating such a scanner in order to acquire the raw MR data. The invention also concerns a non-transitory, computer-readable data storage medium encoded with programming instructions (code) for performing such a method

### Description of the Prior Art

In the context of magnetic resonance examinations, so-called alignment measurements for obtaining anatomical or functional information are carried out before the actual diagnostic data acquisition.

For example, the basic magnetic field is homogenized and the resonance frequency is determined. Automated routines exist for this purpose.

It is further necessary to define the slices or volumes of the examination subject from which MR data are to be acquired. The overview recordings used for this purpose are also referred to as scout scans. This definition of the measurement volume is usually carried out manually, since the placement and number of the measurement slices depends on a multiplicity of conditions related to the framework of the examination, and no automated means of sufficient accuracy are available for all possible questions.

Depending on the measurement parameters and sequence used, distortions can occur in the image data of the alignment measurements in this case. In particular, rapid measurement sequences such as TrueFisp and EPI are susceptible to a wide range of artifacts. Distortions also occur when the scout scans cover volume ranges that extend beyond the isocenter of the magnetic resonance system. The homogenous BO region in the center of the basic magnetic field is designated as the isocenter.

Distortions that occur when using MR methods and that have a negative effect are known in many different contexts. Merely as examples, reference is made to DE 102014210778 B4, DE 102014214844 B4, DE 102014219291 A1 and DE 102015204483 A1.

The distortions are usually corrected automatically by the control computer of the MR apparatus. Therefore, the alignment measurements or other distortedly recorded images cannot be used directly as scout scans, in order to safeguard the positioning accuracy of the measurement volume. The distortion-corrected image data must be redundantly processed and displayed without distortion correction in order to be used for positioning steps.

### SUMMARY OF THE INVENTION

An object of the present invention is to provide a method with which optimum positioning accuracy is possible with minimum computing effort.

This object is achieved by a method for operating a magnetic resonance apparatus that includes the steps of providing a magnetic resonance image data record (file) as an input to a computer, wherein at least one image of the magnetic resonance image data record is distortion-corrected, generating, in the computer, at least one selection symbol for selecting a measurement volume in the image represented by the image data record, wherein the selection symbol is at least partly distorted, and, at a display screen, superimposing the at least partly distorted selection symbol onto a distortion-corrected image of the magnetic resonance image data record.

The displayed distortion-corrected image with the at least partly distorted selection symbol superimposed thereon serves as a user interface that allows a user to manipulate the at least partly distorted selection symbol on the display screen so as to select a measurement region from which diagnostic MR data will be acquired, with control signals corresponding to the selected measurement region then being provided to the MR data acquisition scanner so as to operate the MR data acquisition scanner to acquire the MR raw data for the diagnostic examination from the designated measurement region or volume.

As used herein, “diagnostic MR data” means raw MR data that are acquired in a scan of the subject that are of suitable and sufficient quality so as to reconstruct image data therefrom that are usable for allowing a physician to address the diagnostic issue for which the scan was prescribed. This is in contrast to the MR data that are commonly acquired in the context of a scout scan, which usually are not of sufficient quality or resolution to permit a medical diagnosis to be made from the image data reconstructed therefrom.

The same considerations apply in the context of the acquisition of spectroscopic MR data from a measurement region or volume designated by the at least partly distorted selection symbol.

The basis of the invention is not to recreate the distortion of the image data in the displayed image, but to distort the selection symbol according to the corrected distortion in the image. In this way, the positioning accuracy can be preserved since the volume that is displayed and the volume to be measured correspond again.

The steps for performing the method are preferably executed in a completely automated manner by a control computer of the magnetic resonance system.

By distorting the selection symbol, it is made possible to display the true shape of the examination object during the measurement, this true shape no longer being visible in the distortion-corrected image.

Repeated processing of the images is thereby avoided. Uncertainty is also avoided in this way. If the user sees undistorted images, this may be because the image data contains no distortions since the image depicts the examination region around the isocenter. It is however alternatively possible that the images have been distortion-corrected. Therefore the user no longer has to investigate whether it is first necessary to undo a distortion correction in order to select a measurement volume.

Whether the image used to effect the positioning is distorted only in regions, or everywhere, is revealed by the shape of the selection symbol. The distortion of the selection symbol can be performed by the control computer without significant computing effort and specifically during the positioning of the examination region.

Depending on the underlying magnetic resonance image data record, a single two-dimensional image or a number of two-dimensional images may be present. A three-dimensional image data record is considered to be composed of a number of two-dimensional images in this case. However, a selection symbol is usually positioned on a two-dimensional image. For this purpose, use is either made of images previously recorded in two dimensions or corresponding images are calculated from a three-dimensional image data record. Images are the representation of the image data record in this case.

The difference between an image and an image data record in the context of the invention is as follows. An image data record contains the measurement data that are essential for the reconstruction of one or more images. In the case of parallel imaging, reference is also made to e.g. calibration data from another data record. On the basis of such data, it is possible to effect different post-processing steps in order to generate an image from the image data record. For example, an image data record having 128×128 measurement points may be processed using different zero-filling factors to produce images of 128×128, 256×256 or even 512×512 in size.

The image data record is always the same, but the images differ from each other. The images are what the user sees.

The term “magnetic resonance image data record” is also abbreviated simply as “image data record”.

The distortion of the selection symbol is preferably implemented in a location-dependent (spatially-dependent) manner. The distortion of the image may be location-dependent, e.g. weaker in the center than at the edges. By simulating the distortion of the image or the examination region as accurately as possible, the positioning accuracy of the selection symbol can be optimized.

The selection symbol is preferably distorted such that it has a rectangular cross section after a distortion correction. The cross section is preferably square. The specification for the distortion correction of the image therefore also results in a distortion correction of the selection symbol.

The distortion of the selection symbol is preferably effected as a function of at least one physiological parameter. Physiological parameters are e.g. flow and movement. While flow directions and speeds can be derived from empirical values, movements can be determined using navigators.

The distortion of the selection symbol can be effected as a function of at least one gradient-based parameter, in particular the strength of at least one gradient field. Variations between the reference gradient and the actual gradient lead to incorrect encoding of the spatial positions and hence to distortions. If the variations are known or can be estimated, a corresponding distortion of the selection symbol is also possible.

The distortion of the selection symbol can be effected as a function of the table position of the patient table. The table position can be used as a measure for gradient-based distortions. The distortion is therefore also location-dependent.

The selection symbol is preferably distorted on a line-by-line basis. For example, an individual distortion specification can be provided for each k-space line as a function of the table position. The distortion specification can also vary as a function of the resolution of the magnetic resonance data record. An individual specification can exist for each spatial direction in this case.

The distortion of the selection symbol is preferably effected as a function of at least one physical parameter of the examination object. For example, the so-called chemical shift results in a shift of fat signal relative to water signal. Susceptibility jumps in the examination object act as gradients and therefore likewise lead to incorrect encoding of the spatial information and also to destructive interference of signals.

The distortion of the selection symbol is preferably effected as a function of at least one measurement-sequence-dependent parameter. Such distortions, which are derived from calculable distortions based on the structure of the measurement sequence, can be captured mathematically and therefore simulated particularly accurately.

Depending on the embodiment of the magnetic resonance image data record, distortions of the selection symbol are possible in two or three spatial directions.

The distortion can be effected in the phase encoding direction. Additionally or alternatively, the distortion can be effected in the readout direction. Additionally or alternatively, the distortion can be effected in the slice-selection direction.

Only distortions in two spatial directions can be shown and superimposed on an image in each case. However, multiple individual images, sections, or images of a three-dimensional image data record, can be used in order to define a measurement volume. In this way, it is also effectively possible to take all three spatial directions or gradient directions into consideration.

It is naturally also possible to use images that are not positioned exactly in the readout direction and phase encoding direction or the slice-selection direction. The distortion is then obtained by a projection of the phase encoding direction, readout direction, and slice-selection direction onto the direction of the image.

The distortion of the selection symbol is preferably obtained by an inversion of the distortion correction of the image data record. As previously described, the better the distortion can be described, the greater the increase in the positioning accuracy. If an inversion of the distortion correction is possible, this results in an optimum distortion of the selection symbol.

A grid is preferably used as the selection symbol. Alternatively, a quadrilateral can be used as the selection symbol. Conventional selection symbols are square or at least rectangular. These are transformed by a distortion into less regular quadrilaterals, e.g. a parallelogram.

Regardless of the measurement sequence, the selection symbol always selects a measurement volume, since even in the case of measurements of only a single slice, this has a finite thickness and therefore defines a measurement volume.

The selection symbol can advantageously be used for selecting a measurement volume for a spectroscopic measurement. A measurement volume for a purely spectroscopic measurement can be defined by a quadrilateral, for example, and a measurement volume for a so-called chemical shift image (CSI) can be defined by a grid.

The present invention also encompasses a non-transitory, computer-readable data storage medium encoded with programming instructions (program code) that, when the storage medium is loaded into a computer or computer system of an MR apparatus, cause the MR apparatus to be operated in order to execute any or all of the embodiments of the method according to the invention, as described above.

The invention further concerns a magnetic resonance apparatus having a control computer designed to perform the method as described.

In this case, the implementation of the cited method in the control computer can take place in the form of software or (permanently-wired) hardware.

Further embodiments of the inventive magnetic resonance apparatus correspond to relevant embodiments of the inventive method.

### BRIEF DESCRIPTION OF THE DRAWINGS

FIG. 1 schematically illustrates a magnetic resonance apparatus that is operable in accordance with the invention.

FIG. 2 shows a planning image with a selection symbol (prior art).

FIG. 3 shows a planning image with a selection symbol in accordance with the invention.

### DESCRIPTION OF THE PREFERRED EMBODIMENTS

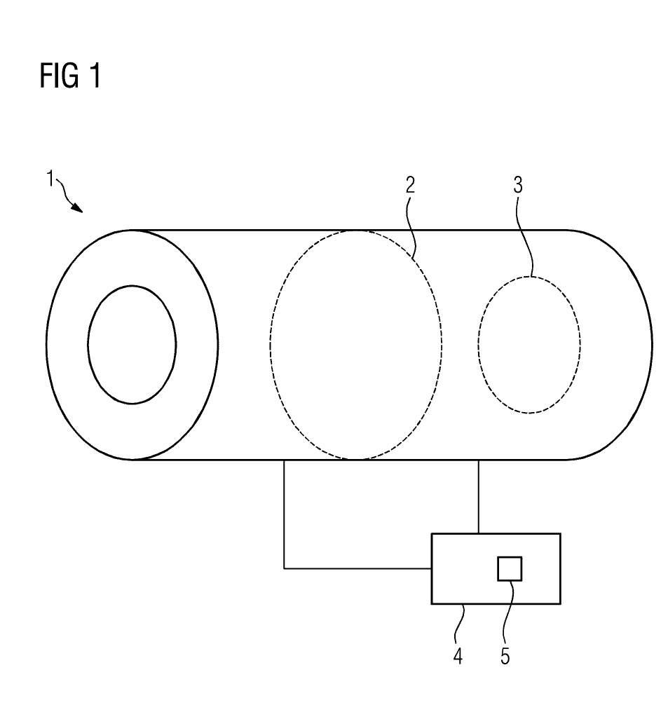

FIG. 1 shows a magnetic resonance apparatus 1, which has a scanner with a transmission coil arrangement 2 and a reception coil arrangement 3. The reception coil arrangement 3 may be designed as a coil array.

A control computer 4 is provided for the purpose of controlling the magnetic resonance apparatus 1, i.e., the scanner thereof.

The magnetic resonance apparatus 1 also has a non-transitory data storage medium 5. The data medium 5 can be designed as part of the control computer 4 or independently thereof. Computer programs for performing magnetic resonance measurements are stored on the data medium 5.

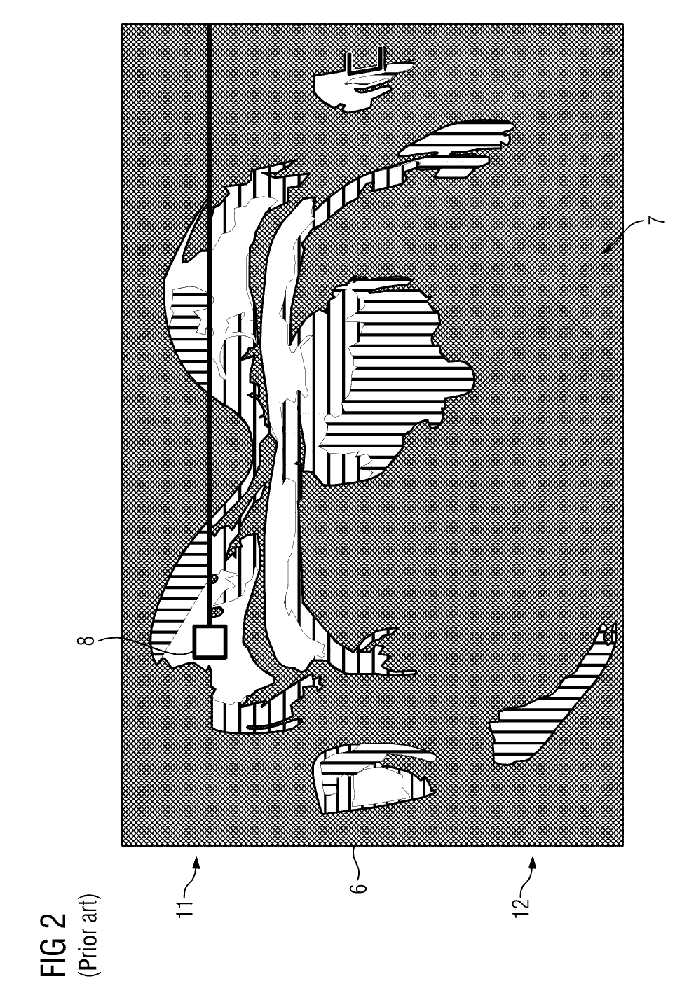

FIG. 2 shows a known planning image 6. The planning image 6 depicts the examination region 7, a cross section at the level of the thorax, on the basis of gradient errors with distortions. Accordingly, the examination region 7 in a first section 11 is more distorted than in a second section 12. The distortions are dependent in particular on the table position of the patient table.

The distortion is shown by example from left to right. This may be situated in read direction, phase direction, slice-selection direction, or a combination thereof. The distortions can occur in all directions.

A selection symbol 8, with which the measurement volume of a spectroscopic measurement can be defined, is superimposed on the planning image 6. The selection symbol 8 is square. If the measurement volume is defined in multiple views, in particular in vertically stacked images, it can also be embodied as a rectangle in another view. The measurement volume is then cuboid or cube-shaped.

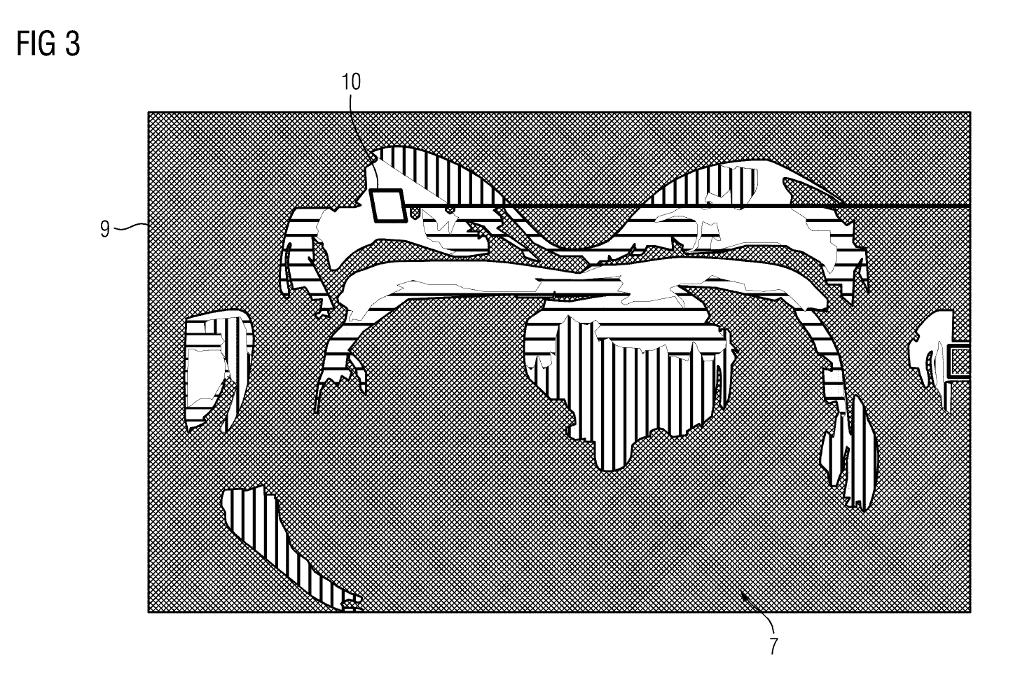

FIG. 3 shows a planning image 9. The planning image 9 was recorded using the same gradient errors as the planning image 6, but it has been distortion-corrected. It therefore resembles an image that was recorded using perfectly constant gradients.

The selection symbol 10 is distorted in accordance with the distortion of the gradient field. A distortion correction of the selection symbol 10 converts this into a square, as shown for the selection symbol 8 in FIG. 2. The same distortion correction also converts the distorted examination region 7 in FIG. 2 into a distortion-corrected examination region 7 and hence into a distortion-corrected planning image 9 as per FIG. 3.

As a result of using a distorted selection symbol 10, it is possible also to use distortion-corrected images as planning images 9 without the positioning accuracy of the measurement volume being adversely affected.

Although modifications and changes may be suggested by those skilled in the art, it is the intention of the Applicant to embody within the patent warranted hereon all changes and modifications as reasonably and properly come within the scope of the Applicant's contribution to the art.

1. A method for operating a magnetic resonance (MR) apparatus comprising:
providing a computer with an MR image data record comprising at least one image of a subject that is distortion-corrected;
in said computer, generating a selection symbol that is at least partly distorted;
at a display screen in communication with said computer, displaying said distortion correction image with said at least partly distorted selection symbol superimposed thereon;
in said computer, accepting a user input made via said display screen resulting from manipulation of said at least partly distorted selection symbol on said distortion-corrected image so as to select a measurement region or measurement volume of the subject from which MR data are to be acquired from the subject; and
in said computer, generating control signals, which include a designation of said measurement volume or said measurement region selected with said at least partly distorted selection symbol, to an MR data acquisition scanner in order to operate the MR data acquisition scanner so as to acquire MR data from the measurement region or measurement volume of the subject.

2. A method as claimed in claim 1 comprising, in said computer, distorting said selection symbol in a spatially-dependent manner.

3. A method as claimed in claim 1 comprising distorting said selection symbol dependent on at least one physiological parameter of the subject, which is provided to said computer as an input.

4. A method as claimed in claim 1 comprising operating said MR data acquisition scanner so as to generate magnetic field gradients during acquisition of said MR data, and distorting said selection symbol dependent on at least one parameter related to said gradients.

5. A method as claimed in claim 1 comprising distorting said selection symbol dependent on at least one physical parameter of the subject, provided to said computer as an input.

6. A method as claimed in claim 1 comprising generating said control signals in said computer so as to cause said MR data acquisition scanner to acquire said MR data by executing a measurement sequence, and distorting said selection symbol dependent on said measurement sequence.

7. A method as claimed in claim 1 wherein said MR data acquisition scanner has a phase direction defined therein, and distorting said selection symbol in said phase direction.

8. A method as claimed in claim 1 wherein said MR data acquisition scanner has a readout direction defined therein, and distorting said selection symbol in said readout direction.

9. A method as claimed in claim 1 wherein said MR data acquisition scanner has a slice-selection direction defined therein, and distorting said selection symbol in said slice-selection direction.

10. A method as claimed in claim 1 wherein said distortion-corrected image is produced by applying a distortion correction, and distorting said selection symbol using an inversion of said distortion correction.

11. A method as claimed in claim 1 comprising representing said selection symbol as a grid.

12. A method as claimed in claim 1 comprising representing said selection symbol as a quadrilateral symbol.

13. A method as claimed in claim 1 comprising generating said control signals in said computer so as to operate said MR data acquisition scanner to acquire said MR data as spectroscopic data from said measurement region or said measurement volume.

14. A non-transitory, computer-readable data storage medium encoded with programming instructions, said storage medium being loaded into a computer of a magnetic resonance (MR) apparatus comprising an MR data acquisition scanner, and said programming instructions causing said computer to:
receive an MR image data record comprising at least one image of a subject that is distortion-corrected;
generate a selection symbol that is at least partly distorted;
at a display screen in communication with said computer, display said distortion correction image with said at least partly distorted selection symbol superimposed thereon;
accept a user input made via said display screen resulting from manipulation of said at least partly distorted selection symbol on said distortion-corrected image so as to select a measurement region or measurement volume of the subject from which MR data are to be acquired from the subject; and
generate control signals, which include a designation of said measurement volume or said measurement region selected with said at least partly distorted selection symbol, to said MR data acquisition scanner in order to operate the MR data acquisition scanner so as to acquire MR data from the measurement region or measurement volume of the subject.

15. A magnetic resonance (MR) apparatus comprising:
an MR data acquisition scanner;
a computer provided with an MR image data record comprising at least one image of a subject that is distortion-corrected;
said computer being configured to generate a selection symbol that is at least partly distorted;
a display screen in communication with said computer, said computer being configured to display said distortion correction image with said at least partly distorted selection symbol superimposed thereon at said display screen;
said computer being configured to accept a user input made via said display screen resulting from manipulation of said at least partly distorted selection symbol on said distortion-corrected image so as to select a measurement region or measurement volume of the subject from which MR data are to be acquired from the subject; and
said computer being configured to generate control signals, which include a designation of said measurement volume or said measurement region selected with said at least partly distorted selection symbol, to said MR data acquisition scanner in order to operate the MR data acquisition scanner so as to acquire MR data from the measurement region or measurement volume of the subject.
